(19) United States
(12) Patent Application Publication
(10) Pub. No.: US 2015/0265546 A1
(43) Pub. Date: Sep. 24, 2015

(54) POLYMERIC NANOCARRIERS WITH DUAL IMAGES TRACKING PROBE AND METHOD FOR MANUFACTURING THE SAME

(71) Applicant: KAOHSIUNG MEDICAL UNIVERSITY, Kaohsiung City (TW)

(72) Inventors: Chih-Kuang Wang, Kaohsiung City (TW); Li-Cheng Pan, Pingtung County (TW); Chih-Yun Lee, Chiayi County (TW); Hong-Jhe Lin, Miaoli County (TW); Che-Wei Lin, Tainan City (TW); Po-Len Liu, Kaohsiung City (TW); Ping-Hsiu Shih, Kaohsiung City (TW)

(73) Assignee: KAOHSIUNG MEDICAL UNIVERSITY, Kaohsiung City (TW)

(21) Appl. No.: 14/662,242

(22) Filed: Mar. 19, 2015

(30) Foreign Application Priority Data
Mar. 21, 2014 (TW) 103110625

(51) Int. Cl. A61K 9/51; A61K 31/7088; A61K 38/18; A61K 51/12; A61K 38/02

(52) U.S. Cl. CPC A61K 9/5153; A61K 9/5169; A61K 9/5161; A61K 51/1251; A61K 38/02; A61K 38/18; A61K 31/7088

(57) ABSTRACT

The present invention relates to a nanoparticle carrier and the method for manufacturing the same. The nanoparticle carrier comprises hydrophobic molecules grafted with nanogold clusters and hydrophobic molecules grafted with hydrophilic molecules. The hydrophilic molecules are located on the outer layer of the nanoparticle and the nanogold clusters are wrapped inside the nanoparticle.

### FIELD OF THE INVENTION

The present invention relates to a target nanocarrier that possesses high biocompatibility, is capable of efficiently encapsulating and carrying active materials and can be tracked effectively.

### BACKGROUND OF THE INVENTION

Many polymeric materials can be used as drugs and drug carriers in current medical and pharmaceutical research. Since most polymeric materials when applied into the living bodies tend to be identified and processed as foreign objects, subsequently triggering immune response, the applicable polymeric materials are rare. Moreover, some drugs cannot be delivered directly into the living bodies by injection or oral administration, such type of drugs are usually manufactured in combination with compounds. Among them, polymeric materials account for a relatively higher proportion. Two requirements are generally evaluated when assessing drug delivery of biopo lymeric materials: bio compatibility and biodegradability. Bio compatibility refers to the condition where biomaterials are compatible to living bodies without developing rejection effects. They should not induce or develop thrombosis when applied, and should be non-toxic, non-allergenic, non-inflammatory, free of immune response, non-carcinogenic and non-tissue destructive. Biodegradability refers to the condition when biomedical polymeric materials are inside the living organisms where under reactions, such as enzymes, body fluids, hydrolysis and oxidation, the integrity of the polymers is destructed to yield fragments or degraded into other products that can be degraded and excreted off the body via basic metabolism in vivo.

Generally, the polymeric materials for medical and pharmaceutical use can be categorized into two classes. One is natural polymeric materials, and the other is synthetic polymeric materials. The artificial synthetic materials commonly used as a drug carrier include polyesters (PE), such as commonly seen polylactide (PLA), polyglycolide (PGA) or poly(lactide-co-glycolide)(PLGA), and other different types of artificial synthetic polymeric materials, such as polyanhydrides, mostly for textile use, polyethylene glycol (PEG) and poly ethylene oxide (PEO), the most commonly seen polyether. The most commonly seen natural polymeric materials are materials such as chitosan, chitin and collagen. Majority of the commonly seen drug carriers are used in combination with different copolymers to compensate each other for better effects.

The use of polymers as the materials for drug carriers has been developed and applied for several years. For designing the drug carriers, the polymeric materials described above for biological applications can be modified or combined to form polymeric structure carrying multiple functional groups. Through different types of applications the multiple functional groups can be modified or designed to carry and encapsulate drugs, such as proteins, target antibodies, oil soluble or degradable drugs, thus the drug carriers can not only be able to carry and deliver the drugs, but also protect and preserve the drug function as well as amino acid-specific targeting potential of the antibodies for specific diseased part, resulting in dosage and toxicity reduction to reach effective therapeutic goals.

The natural biological materials possess the advantage of absolute biocompatibility, an aliphatic polyester is one of commonly seen biodegradable polymeric materials, including PLA, PGA and PLGA. Aliphatic polyesters possess the following advantages: (1) polyesters are widely used as materials in clinical application. In 1970s, the United State FDA approved use of PLGA in surgical suture materials. In the recent years it has been widely used in manufacturing cytoskeletons or templates. (2) non-toxic. Lactic acid and glycolic acid generated through hydrolysis of such polymers can be converted into carbondioxide and water molecules via normal physiologic metabolic process (tricarboxylic acid cycle) and excreted out of body without residual inside human body. (3) degradation rate is controllable. Except the two formers, PLGA, through modification of monomer molar ratio, molecular weight, crystallinity, pH value and enzymes (esterases) of the copolymers, can be modified to yield polymers with different degradation rates, it is easy to be designed, controlled and matched with the regeneration rate of the newly formed tissues.

PEG is synthesized from polymerization of ethylene oxide, composed of repetitive vinyl oxide, not only with good water solubility but also soluble in organic solvent such as dichloromethane, N′N′-dimethylformamide, benzol, acetonitrile and ethanol, and has linear (5,000-30,000 Da relative molecular weight) or branching (40,000-60,000 Da relative molecular weight) chain structure, the molecular formula for linear PEG is H—(O—CH2—CH2)n-OH. Each of the two ends of common PEG contains a hydroxyl group, mPEG could be yielded if one end is capped with methyl. The molecular formula for linear mPEG is CH3-(O—CH2-CH2)n-OH. In research of PEG modification of polypeptides and proteins, mPEG derivatives are the most used. As for physiological characteristics, PEG is neutral, non-toxic, with unique physiochemical property, a polymer with good biocompatibility and one of the rare synthetic polymers approved by FDA for the use of internal injection. The pharmacokinetic characteristics of the PEG modifiers are different from each other depending on their relative molecular weights and routes of injection administration with longer half-life for higher molecular weight. Through oxidation by cytochrome P450 system, PEG is fragmented into small-molecule PEG and excreted via bile.

Molecular imaging mainly utilizes imaging methods to display specific molecular level of tissues, cells and subcellular structures. The commonly used imaging equipment includes ultrasound, computed tomography (CT), magnetic resonance imaging (MRI) and positron emission tomography (PET).

(1) Ultrasound:

Wave motions above 20,000 Hz frequency in the sound wave are utilized and converted into images for tracking, usually used in obstetrics and gynecology or internal evaluation.

(2) Computed Tomography (CT):

Computed tomography (CT) belongs to cross-sectional imaging equipment, initially used for examining human brain structures. The principle is to use X-ray to pass through the objects to be examined from multiple angles and to observe the reduction of the energy during penetration, followed by conversion into current signals which can be processed to generate recognizable images. It allows healthcare professionals to see the structural changes of various internal organs inside the human body via non-invasive methods, providing significant benefits in disease diagnosis. A general CT system uses X-ray as the source of energy. Different properties of human tissues can be detected using different types of energy sources, thus different types of CT images are developed. Sometimes for more accurate results in examination, the examination would be proceeded in combination with injection of CT developing agents. The developing agents through combination of iodine and some macromolecules are capable of blocking penetration of X-ray. Sometimes we also call the developing agents as “contrast agents” which generate contrast effects by using the differences in absorbance of developing agents between abnormal and normal tissues, leading to improvement of diagnostic results.

(3) Magnetic Resonance Imaging (MRI):

Currently magnetic resonance imaging (MRI) is a relatively newer medical imaging technology. Its principle comes from nuclear magnetic resonance (NMR) where the human subject is placed in a strong and even magnetic field during general scanning process and specific radiofrequency (RF) pulse is used to excite hydrogen atoms inside the human tissues to generate resonance phenomena, yielding and receiving signals from the changes of magnetic moment which then are collected and transformed into 3-dimensional images.

(4) Positron Emission Tomography (PET):

Positron Emission Tomography (PET) is a computed tomography mainly used for detecting positron. The positron is the antiparticle of the electron where different from the electron it carries positive electric charge. When practically proceeding imaging examination, the positron-emitting isotope tag compounds have to first be administered into the testing body intravenously, per os or through inhalation. After being injected into the body this agent continuously decays to generate positrons, the positrons inside the human body rapidly collide with the electrons in the body, generating annihilation that produces a pair of gamma rays (energy) moving in opposite directions. The gamma rays possess very high energy, powerful enough to penetrate human body, thus an external detector can be used to detect this pair of photons and to track the location of radiopharmaceuticals in the body. The positron emission area is the source of the signals in the body, and also the location of image produced.

In early research, some organic compounds emitting fluorescence, like fluorescein isothiocyanate (FITC), were commonly used as an agent for observing the cells or the animals in vivo. But not all the organic compounds are suitable for every kind of experiment, mainly owing to the partial toxicity to the live cells and animals from the chemical property of the organic compounds. Hence agents with lower toxicity are preferred during most experimental processes. Another disadvantage is the difficulty of preserving the organic compounds where light exposure should be avoided to reduce loss of fluorescence. To improve the limitation of the organic compounds, biocompatible materials with fluorescent property and low toxicity are being sought in the recent years to replace the organic compounds currently used in fluorescent experiments. Development and application of the fluorescent gold nanomaterials becomes one of the improvement strategies. Taking advantages of high biocompatibility and non-toxic property of gold, research and development is proceeded to reduce its size and enhance its surface area. Compared with currently used metal materials, significant difference exists in optics, magnetism, electricity and catalytic property. The mechanism of fluorescence emission by gold nanoclusters (AuNCs) comes from the surface plasmon resonance produced by enhanced phenomenon of collective excitation of electrons when the average size of AuNCs reaches the mean free path of electrons (about 50 nm) (Tang, L.; Azzi, J.; Kwon, M.; Mounayar, M.; Tong, R.; Yin, Q.; Moore, R.; Skartsis, N.; Fan, T. M.; Abdi, R.; Cheng, J., Immunosuppressive activity of size-controlled PEG-PLGA nanoparticles containing encapsulated cyclosporine A. J. Transplant. 2012, 896141, 9 pp). To synthesize AuNCs with single-layer thiolate surface modification, a gold ion precursor, such as HAuCL4 or AuBr, is usually combined with reducing agent NaBH4 and thiolate under basic environment, and with specific ratio the above step reduces gold ions to form AuNCs with thiolate modified surface. Using different thiolates, the optic property and emission wave band yielded are different. Most AuNCs have emission wave band around 600 nm, plus the lower background noise from biomatrix beyond 600 nm, it is therefore advantageous to use AuNCs as a tracking tool in examination of living organisms (Brust, M.; Walker, M.; Bethell, D.; Schiffrin, D. J.; Whyman, R., Synthesis of thiol-derivatized gold nanoparticles in a two-phase liquid-liquid system. J. Chem. Soc., Chem. Commun. 1994, (7), 801-2).

More and more cancerous or cellular abnormality-related disorders have been discovered in the recent years and many drugs have been derived from the respective therapeutic regiments. The effects of commonly seen therapeutic drugs on human body are biphasic, not only toxic to the abnormal sites, but also affecting the normal cells and organs. Therefore, development of novel drug carriers for this problem is needed to resolve the unnecessary damage against human body by the drugs and to increase accuracy of drug delivery, leading to effective drug delivery and therapy. Various development of drug carriers in recent years are focused on improvement of toxicity to normal cells generated by conventional chemotherapeutic drugs and on reduction of drugs used, and together with target therapy model are designed to create targeting drug carriers. This idea strengthens the mechanism of controlled drug release providing safer choice of cancer treatment (Yu, M. K.; Park, J.; Jon, S., Targeting strategies for multifunctional nanoparticles in cancer imaging and therapy. Theranostics 2012, 2 (1), 3-44).

In nanodrug carrier research, majority of nanoparticles are found to accumulate in the tumor tissues. The cause of this pathological characteristic comes from that cancerous tissues when larger than 2 mm in size during the course of cell division would secret proangiogenic factors to develop new blood vessels to provide necessary nutrition and oxygen under the stress of limited nutrition and the need of continuous growing. The intercellular space among the newly generated blood vessels ranges from about 100 nm to 2 μm, which is larger than the normal intercellular space, thus causing a large portion of nutrition to be lost easily. Designing the nanoparticles to have a particle diameter smaller than the intercellular space in the tumor, through circulation of the blood, can effectively cause nanoparticles to stop and accumulate in the tumor tissues. The function described above is called enhanced permeability and retention effect (EPR effect), resulting in passive targeting and selective accumulation (Acharya, S.; Sahoo, S. K., PLGA nanoparticles containing various anticancer agents and tumour delivery by EPR effect. Adv. Drug Delivery Rev. 2011, 63 (3), 170-183). Hence studying nanoparticles in tumor intercellular space becomes one of the important mechanisms in recent years.

Despite specific effect of passive targeting, the methods of passive targeting have certain limitation and utilization of EPR effect cannot be applied on all types of cancer cells owing to various sizes of intercellular space in differential tumor types and conditions. Lack of accuracy and controllability may cause loss of drugs and induce cancer cells to become drug-resistant, leading to inevitable reduction of therapeutic effects. To improve disadvantage of limitation of passive targeting system, targeting capabilities are added to the surface of nanoparticles and this system is generally called “active targeting” which through ligand-receptor interaction can bind to the target cells, causing receptor-induced endocytosis and intracellular drug release. This drug delivery strategy realizes specific binding and enhances drug delivery effectiveness while precluding non-specific binding and drug-resistant development. According to the clinical trials, some targeted delivery systems, such as CALAA-01 and MBP426, are currently available. The potential of ligand-led targeting technology combined with capability of nanodrug carrier has become the focus of different research studies.

Cancer or related disorders have been seen more and more frequently. Finding the cause of disorders has always been the best direction. Early cancer treatment focused on surgical removal of the abnormal tissues. However, many problematic conditions evolve from the surgery, for example, problems like the post-surgical cancer metastasis and that the patients with late-stage cancer are too weak to go through the surgery. Some compounds with therapeutic toxicity have been generated through recent discovery and study by the researchers. Although the compounds can partially poison cancer cells, they also damage the normal cells and organ function. To reach effective poisoning effect, high dosage is often given, leading to discomfort and nausea on the patients, a big challenge in cancer treatment. To decrease the damage to human body by the drugs, reduction of the drug dosage while providing effective therapeutic poisoning effect is the first approach to reduce the discomfort and panic of the patients. Toward this direction, many drug carriers have been developed to encapsulate drugs for entering human body and reaching their effects. Poisoning cancer cells can be done by drug therapy and imaging methods are needed to observe and to check if the drugs can reduce the size of the tumor. Sometimes contrast agents are injected for the need of effectiveness in different observation methods during observation process, often causing additional damage to the patients. Therefore, a target drug carrier that is highly biocompatible and capable of encapsulating and carrying drugs efficiently and can be tracked effectively is needed.

Although Mieszawska et al. have synthesized PLGA nanospheres modified with AuNCs (Mieszawska, A. J.; Gianella, A.; Cormode, D. P.; Zhao, Y.; Meijerink, A.; Langer, R.; Farokhzad, O. C.; Fayad, Z. A.; Mulder, W. J. M., Engineering of lipid-coated PLGA nanoparticles with a tunable payload of diagnostically active nanocrystals for medical imaging. Chem. Commun. (Cambridge, U. K.)2012, 48 (47), 5835-5837), the PEG is dispersed on the surface of the nanospheres through coating without chemical bonds with PLGA, yielding a disadvantage in its design and resulting in unstable structure that causes detachment from the nanospheres easily. Therefore, Mieszawska's PLGA nanosphere cannot be used as a target carrier because that it's efficiency of ligating target molecules is not good. In addition, quantum dots (QDs) need to be used as a source of fluorescence during its design.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is the schematic diagram of AuNC synthesis reaction.

FIG. 2 is the schematic diagram of PLGA-mPEG synthesis reaction.

FIG. 3 is the schematic diagram of PLGA-AuNCs synthesis reaction.

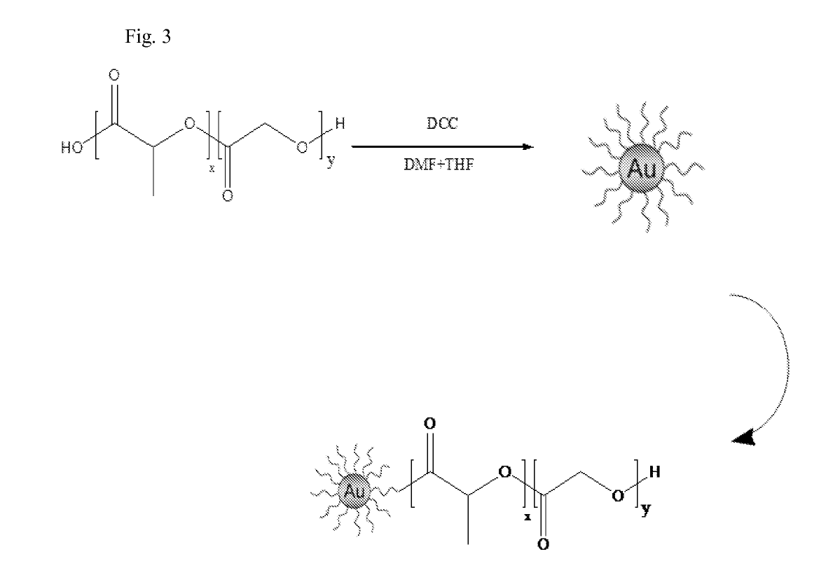

FIG. 4 is the schematic diagram of nanotechnology.

FIG. 5 shows the results of analysis ofAuNCs using fluorescence spectrometer.

FIG. 6 is the TEM image of AuNCs.

FIG. 7 is the fluorescent spectrum of PLGA and PLGA-mPEG nanoparticles.

FIG. 8 is the fluorescent spectrum of the composite nanoparticles.

FIG. 9 is the MTS assay result of the effect of AuNCs on the viability of HeLa cell line.

FIG. 10 is the MTS assay result of the effect of AuNCs on the viability of 3T3 cell line.

FIG. 11 is the MTS assay result of the effect of the composite nanoparticle on the viability of HeLa cell line. Numbers 1 to 5 represent groups of PLGA, PLGA-mPEG, PLGA-AuNCs:PLGA-mPEG (2:1), PLGA-AuNCs:PLGA-mPEG (1:1) and PLGA-AuNCs:PLGA-mPEG (1:2) respectively.

FIG. 12 is the MTS assay result of the effect of the composite nanoparticle on the viability of 3T3 cell line. Numbers 1 to 5 represent groups of PLGA, PLGA-mPEG, PLGA-AuNCs:PLGA-mPEG (2:1), PLGA-AuNCs:PLGA-mPEG (1:1) and PLGA-AuNCs:PLGA-mPEG (1:2) respectively.

FIG. 13 is the test result of binding specificity of PLGA-mPEG nanoparticle against anti-PEG antibody.

FIG. 14 is the test result of binding specificity of PLGA-AuNCs:PLGA-mPEG (1:1) nanoparticle against anti-PEG antibody.

FIG. 15 is the fluorescent images of the PLGA nanoparticles encapsulating FITC in endocytosis assay on HeLa cells without (A) and with (B) anti-PEG antibody.

FIG. 16 is the fluorescent images of the PLGA-AuNCs:PLGA-mPEG (1:2) nanoparticles encapsulating FITC in endocytosis assay on HeLa cells without (A) and with (B) anti-PEG antibody.

FIG. 17 is the fluorescent images of the PLGA-AuNCs:PLGA-mPEG (1:1) nanoparticles encapsulating FITC in endocytosis assay on HeLa cells without (A) and with (B) anti-PEG antibody.

FIG. 18 is the fluorescent image of live animals.

FIG. 19 is the micro CT image of AuNCs and PLGA-AuNCs:PLGA-mPEG (1:1) nanoparticles.

FIG. 20 is the schematic diagram of the nanoparticle carrier of the present invention.

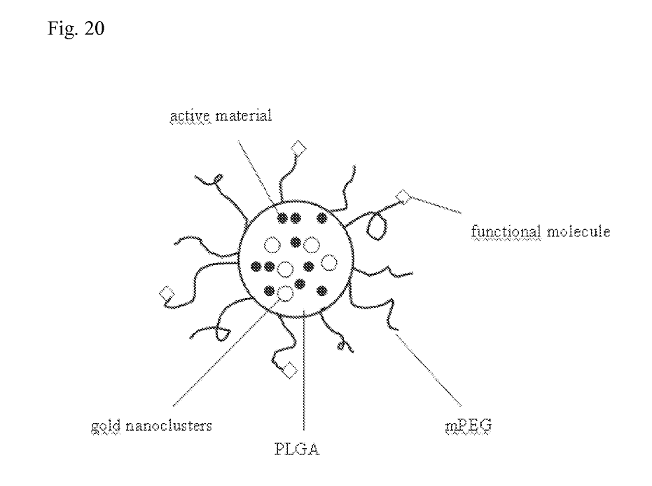

FIG. 21 is the flowchart of PC5-2 peptide modification of the composite nanoparticle surface.

FIG. 22 is the test result of PC5-2 peptide BCA.

FIG. 23 is the result of in vitro cellular endocytosis assessment of the PC5-2 peptide through fluorescent microscope. (A): A549 cell line; (B): 3T3 cell line.

FIG. 24 is the result of in vivo image assessment of PC5-2 peptide target tracking.

### SUMMARY OF THE INVENTION

The present invention relates to a nanoparticle carrier comprising hydrophobic molecules bonded with gold nanoclusters (AuNCs) and hydrophobic molecules bonded with hydrophilic molecules, wherein the hydrophilic molecules are located in the outer layer of the nanoparticle and the AuNCs are encapsulated inside the nanoparticle. The present invention also relates to a method of manufacturing the nanoparticle carrier described above, comprising (a) dissolving hydrophobic molecules bonded with gold nanoclusters (AuNCs) and hydrophobic molecules bonded with hydrophilic molecules in an organic solvent to yield a mixture; and (b) adding water into the mixture.

### DETAILED DESCRIPTION OF THE INVENTION

The objective of the present invention is to manufacture a target drug carrier with high biocompatibility, capable of efficiently encapsulating and carrying drugs that could be tracked effectively. The method is to utilize PLGA as a backbone to bind the AuNCs prepared with (±)-α-diaphorase ((±)-α-Lipoamide) as the main structure, synthesizing PLGA-AuNCs, followed by combination with PEG-PLGA, that are synthesized by binding hydrophilic PEG onto the same PLGA-based main structure, to form nanoparticles. Hydrophilicity of PEG is utilized to increase circulation period of the nanocarrier inside the body in order to increase the length of drug releasing time. Two different PLGA nanoparticles with different modifications are combined in various ratios in response to the purposes needed, resulting in efficiently encapsulating and carrying the drugs and effectively tracking the drugs and observing the therapeutic process by utilizing the high biocompatibility and fluorescent property of the AuNCs.

The present invention relates to the multi-functioning cancer target nanocarrier, i.e., the nanocarrier having functions of carrying anti-cancer drugs, targeting tumor, and image tracking of the nanocarrier at the same time. The composition of this cancer target nanocarrier includes PLGA-AuNC formed by grafting the AuNCs with near-IR fluorescence and CT image tracking system onto the end of poly(lactic-co-glycolic acid) (PLGA) copolymer and the 2-part PLGA-mPEG copolymer synthesized by grafting PEG onto PLGA, mixed in different ratios of these two to separate out composite nanoparticles. Therefore, the goals of the present invention include (1) synthesizing and analyzing the AuNCs with near-infrared. (2) synthesizing and validating AuNC-grafted PLGA. (3) synthesizing and validating PEG-grafted PLGA. (4) mixing PLGA-AuNC and PLGA-mPEG in different ratio and separating out composite nanoparticles, and evaluating the properties of the nanoparticles and their capability of encapsulating and carrying drugs. (5) evaluating the image tracking potential of this multi-functioning cancer target naocarrier and evaluating the specifically targeting ability of PEG against anti-PEG antibody. (6) modifying the nanoparticle surface with targeting capability molecule, PC5-2 peptide, and through image tracking observing its active targeting ability against A549 (human lung carcinoma cell line) and 3T3 cell lines. The near-infrared fluorescent AuNC is synthesized using (±)-α-diaphorase as a template as well as the amine at the end of (±)-α-diaphorase and the carboxyl group at the end of PLGA are bound to form an amide bond for grafting reaction. The chemical structures of the synthesized PLGA-AuNC and PLGA-mPEG are evaluated using nuclear magnetic resonance (NMR) and Fourier transform infrared spectroscopy (FTIR), the distribution of nanoparticle size is analyzed using ζ potential-particle size analyzer, the composite nanoparticle pattern is observed using transmission electron microscope (TEM). In vitro cellular analysis is proceeded with MTT assay to investigate the toxicity in HeLa and 3T3 cell lines. Cancer cell endocytosis assay is proceeded using HeLa3 cell line. Endocytosis assay is designed to encapsulate a FITC as a modeling drug and through laser scanning confocal microscopy (LSCM) to observe the fluorescent signals of AuNC and FITC fluorescence as well as endocytosis property, ensuring that composite nanoparticles are capable of encapsulating the lipophilic drugs and tracking and evaluating the fluorescent of infrared. In vivo studies is done by utilizing live molecular imaging system (Caliper IVIS system) and injecting this composite nanoparticles into the mouse body to observe the fluorescence of the AuNC and to assess CT imaging potential. The surface of nanoparticles is modified with the active targeting molecule, PC5-2 peptide, A549 cell line and 3T3 cell line are used for endocytosis assay, through LSCM the fluorescent signals and cellular endocytotic characteristics are observed to confirm that the composite nanoparticle possesses capability of active targeting as well as near-infrared fluorescence tracking assessment. As for in vivo fluorescent image tracking, the composite nanoparticle modified with PC5-2 peptide is injected subcutaneously into the A549 cell line-generated tumor carrying nude mice for observation of its targeting ability and comparison with the control composite nanoparticle without PC5-2 modification. The initial results indicate correct chemical structures of 2 synthetic PLGA-AuNC and PLGA-mPEG confirmed by NMR spectrum. Particle size analysis shows the size of this composite nanoparticle ranging from 100 to 120 nm. AuNC is indeed distributed in the composite nanoparticle via TEM observation. Cellular MTT assay indicates that this composite nanoparticle is non-toxic to cells. Through confocal microscopy observation it is confirmed that this composite nanoparticle is capable of carrying lipophilic drug fluorescence into HeLa cells with AuNC being trackable by infrared. This result indicates high feasibility of reaching the goals of cancer cell therapy in the future. IVIS live fluorescent imaging observation confirms that this composite nanoparticle possesses live cancer PEG antibody targeting potential in its fluorescence inside the body and near-infrared tracking imaging potential. As for CT imaging contrastable property, capability of being contrasted is shown in high concentration in vitro. Through confocal microscopy observation of the PC5-2 peptide-modified composite nanoparticles capable of active targeting, it is found that the particles can enter to the cytoplasm of A549 cells within 5 minutes, faster than the nanoparticles without target molecule-modification. This result proves that target molecules can promote the nanoparticle in cancer therapy to be faster and more accurate while reducing side effects and damage against caused during drug administration. Utilizing IVIS animal fluorescent image tracking, subcutaneous injection is proceeded on the A549 cell line-generated tumor model nude mice, it is found in the images that composite nanoparticles modified with PC5-2 molecule can rapidly accumulate at the tumor site. The present invention successfully synthesize highly biocompatible and multi-functional cancer target nanocarrier capable of carrying lipophilic drugs, PEG antibody tumor targeting, active targeting by target molecule modification, with infrared and CT imaging tracking potential for the nano carrier.

Unless otherwise defined in the present context, the scientific and technical terms used in the present invention should possess meaning commonly known by any person with ordinary skill in the art. The meaning and scope of the terms should be clear; nevertheless, in any circumstance of discrepancy in the meaning, definition provided in the present context precedes those defined in any other dictionaries or external references.

The entire context of any references cited in the present context is incorporated into the present context as references.

Unless otherwise needed in the present context, singular terms should include plural forms and plural terms should include singular forms.

Hence, the present invention provides a nanoparticle carrier with function in both infrared and CT imaging tracking, which comprises hydrophobic molecules bonded with gold nanoclusters (AuNCs) and hydrophobic molecules bonded with hydrophilic molecules, wherein the hydrophilic molecules are located in the outer layer of the nanoparticle and the AuNCs are encapsulated inside the nanoparticle. The hydrophobic molecules may be, for example, poly(lactide-co-glycolide) (PLGA), polycaprolactone (PCL), polyvalerolactone (PVL), polylactic acid (PLA), polybutyrolactone (PBL), polyglycolide (PLG), or polypropiolactone (PPL); the hydrophilic molecules may be, for example, polyethylene glycol (PEG), hyaluronic acid, poly(glutamic acid) (PGA), dextran, chitosan, or gelatin. In one embodiment, the hydrophobic molecules bonded with AuNCs are PLGA-AuNCs and the hydrophobic molecules bonded with hydrophilic molecules are PLGA-mPEG. Generally, the particle size of the nanoparticle ranges from 20 to 300 nm; in one embodiment, the particle size of the nanoparticle ranges from 90 to 140 nm. The nanoparticle carrier of the present invention can further encapsulate an active material inside. The active material may be, for example, drugs, proteins, polysaccharides, radioactive substances, growth factors, or genes, wherein the preferred drugs are lipophilic drugs. The hydrophilic molecules in the outer layer of the nanoparticle carriers of the present invention may be further bonded with a functional molecule, wherein the functional molecule may be, for example, a targeting molecule with targeting capability.

The present invention also provides a method of manufacturing the nanoparticle carrier described above, comprising (a) dissolving hydrophobic molecules bonded with gold nanoclusters (AuNCs) and hydrophobic molecules bonded with hydrophilic molecules in an organic solvent to yield a mixture; and (b) adding water into the mixture. The hydrophobic molecules may be, for example, poly(lactide-co-glyco lide) (PLGA), polycapro lactone (PCL), polyvalero lactone (PVL), polylactic acid (PLA), polybutyrolactone (PBL), polyglycolide (PLG), or polypropiolactone (PPL); the hydrophilic molecules may be, for example, polyethylene glycol (PEG), hyaluronic acid, poly(glutamic acid) (PGA), dextran, chitosan, or gelatin. In one embodiment, the hydrophobic molecules bonded with AuNCs are PLGA-AuNCs and the hydrophobic molecules bonded with hydrophilic molecules are PLGA-mPEG. The mixing ratio of PLGA-AuNCs and PLGA-mPEG may range from, for example, about 1:10 to 10:1 or about 1:5 to 5:1; in one embodiment, the mixing ratio of PLGA-AuNCs and PLGA-mPEG ranges from about 1:2 to 2:1. The organic solvent in the method may be, for example, dimethylformamide (DMF), dimethyl sulfoxide (DMSO), tetrahydrofuran (THF), acetone, dichloromethane, or chloroform. In the method, the ratio of the water and the organic solvent in volume may be, for example, about 1:20 to 20:1; in one embodiment, the ratio of the water and the organic solvent in volume is about 4:1. In the method, step (a) may further comprise addition of an active material into the mixture. The active material may be, for example, drugs, proteins, polysaccharides, radioactive substances, growth factors, or genes, wherein the preferred drugs are lipophilic drugs. Moreover, the hydrophilic molecules in step (a) in the method may be further bonded with a functional molecule, the functional molecule may be, for example, a targeting molecule with targeting capability.

### EXAMPLES

The examples below are non-limiting and are merely representative of various aspects and features of the present invention.

### Example 1

### Materials and Methods:

### Method of Preparing AuNCs

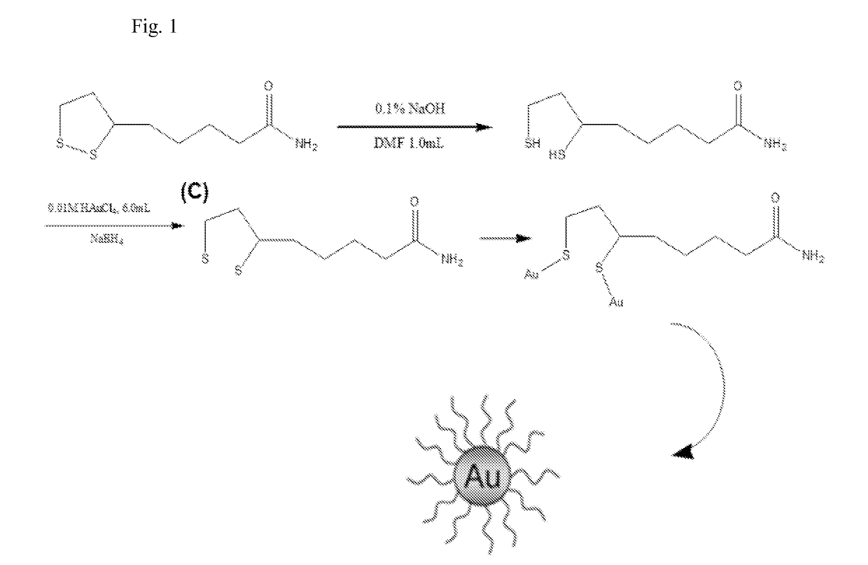

To synthesize AuNC with surface modification with single layer of thiamine molecules, the present method referred to the method described in Shang, L.; Azadfar, N.; Stockmar, F.; Send, W.; Trouillet, V.; Bruns, M.; Gerthsen, D.; Nienhaus, G. U., One-Pot Synthesis of Near-Infrared Fluorescent Gold Clusters for Cellular Fluorescence Lifetime Imaging. Small 2011, 7 (18), 2614-2620. Utilizing (±)-α-diaphorase as the main molecule, appropriate amount of (±)-α-diaphorase (purchased from Sigma-Aldrich) was taken and disolved in dimethylformamide (DMF) solvent, then transferred into the reaction flask. Prepared NaOH solution (purchased from J. T. Baker) was added slowly, stirred for 5 to 60 minutes to complete open-loop of (±)-α-diaphorase. After completion of open-loop using basic solution, prepared gold(III) chloride trihydrate (HAuCl4) (purchased from Sigma-Aldrich) was slowly added in droplet in the same way, stirred for 5 minutes, then the reaction flask was transferred to an ultrasound oscillator to proceed oscillation at 4° C. . NaBH4 (purchased from Sigma-Aldrich) was added slowly in droplet during oscillation. Here oscillation was used to proceed reaction mainly to mitigate redox reaction developed rapidly during addition of NaBH4 and to prevent AuNCs from fast aggregation which formed over-sized particles that would lead to experimental failure. After completion of this step, the reaction flask was removed from the ultrasound oscillator and stirred for 10 to 60 minutes for reaction using a general magnetic stirrer. After completion of the reaction, equal volume of methanol was added, a decompression concentrator was used to proceed concentration, DMF dialysis was proceeded for 2 to 4 days to yield AuNCs with surface modified with (±)-α-diaphorase. The schematic diagram of the synthesis reaction described above was shown in FIG. 1.

Method of preparing PLGA-mPEG

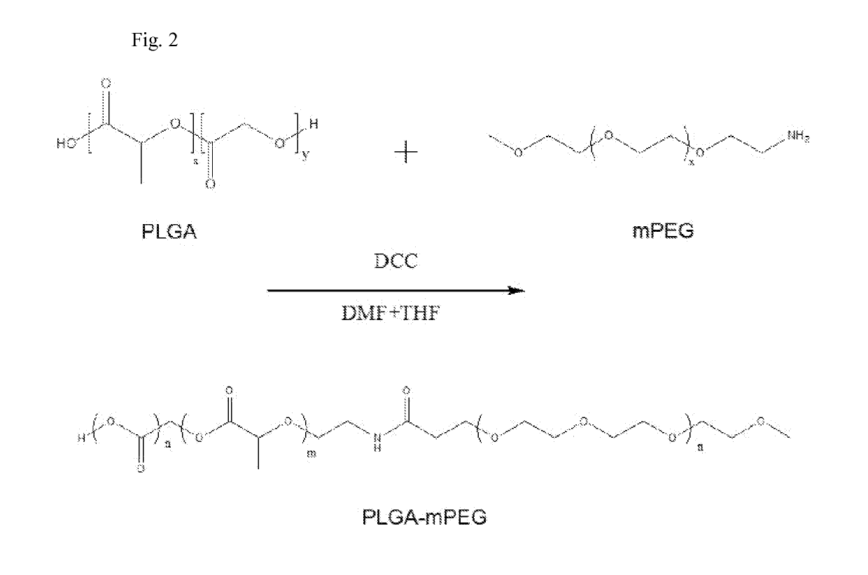

The material used in the present method was polyethylene glycol with methoxy at one end and amine at the other end as bifunctional groups and with molecular weight of approximately 2015 Dalton. PLGA with L-lactic acid:glycolic acid=50:50 (purchased from Sigma) was used as another material with its molecular weight ranging from 7,000 to 17,000 Dalton. During the experiment mPEG, PLGA and N,N′-Dicyclohexylcarbodiimide (DCC) were taken to proceed amidation reaction with tetrahydrofuran/dimethylformamide organic solvent to yield PLGA-mPEG di-block copolymer product. The preparing method referred to the method described in Saadati, R.; Dadashzadeh, S.; Abbasian, Z.; Soleimanjahi, H., Accelerated Blood Clearance of PEGylated PLGA Nanoparticles Following Repeated Injections: Effects of Polymer Dose, PEG Coating, and Encapsulated Anticancer Drug. Pharm. Res. 2013,30 (4), 985-995. Using PLGA as the base material, PLGA and N,N′-Dicyclohexylcarbodiimide (DCC, molecular weight=206.33) were taken and dissolved in dimethylformamide (DMF): tetrahydrofuran(THF) organic solvent, stirred for 6 to 12 hours for reaction, then OMe-PEG-H2N (molecular weight=2015, purchased from Laysan Bio Inc.) was added and stirred for 6 to 12 hours for amidation reaction. Purification was proceeded after reaction was completed, the solution was transferred into the dialysis bag with molecular weight cut-off (MWCO) of 12,000, DMF was used for dialysis for 1 day, followed by 3 to 4 days of dialysis using double-distilled water. After dialysis, freeze-dry method was used to obtain PLGA-mPEG in white powder. The schematic diagram of the synthesis reaction described above was shown in FIG. 2.

### Method of Preparing PLGA-AuNCs

L-lactic acid:glycolic acid=50:50 (purchased from Sigma) was used in the present method with molecular weight ranging from 7,000 to 17,000 Dalton. 300-600 mg PLGA was added into AuNC suspension solution during the experiment, DCC was used to proceed amidation reaction with THF/DMF organic solvent to yield PLGA-AuNCs copolymer product. The preparing method referred to the method described in Mieszawska, A. J.; Gianella, A.; Cormode, D. P.; Zhao, Y.; Meijerink, A.; Langer, R.; Farokhzad, 0. C.; Fayad, Z. A.; Mulder, W. J. M., Engineering of lipid-coated PLGA nanoparticles with a tunable payload of diagnostically active nanocrystals for medical imaging. Chem. Commun. (Cambridge, U. K.) 2012, 48 (47), 5835-5837. Using PLGA as the base material, PLGA and DCC were dissolved in DMF:THF, stirred for 6 to 12 hours for reaction, then about 100 μl of AuNCs with surface modified with (±)-α-diaphorase was added and stirred for 6 to 12 hours for amidation reaction. Purification was proceeded after reaction was completed, the solution was transferred into the dialysis bag with molecular weight cut-off (MWCO) of 12,000, DMF was used for dialysis for 1 to 3 days, followed by 3 to 4 days of dialysis using double-distilled water. After dialysis, freeze-dry method was used to obtain PLGA-AuNCs in light-brown white powder.

Surface Modification of Composite Nanoparticles with PC5-2 Peptide

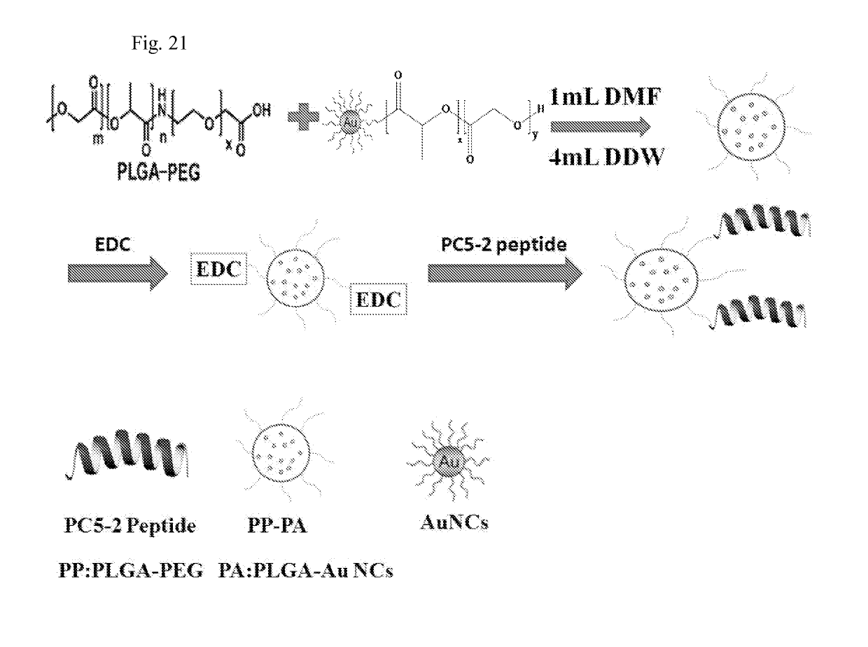

The two materilas described above, PLGA-PEG and PLGA-AuNCs, were used, 25 mg was taken and mixed with defined ratio to form nanoparticles. PC5-2 peptide solution was added and N-(3-Dimethylaminopropyl)-N′-ethylcarbodiimide hydrochloride (EDC) was used to proceed amidation reaction in DD water to modify the nanoparticle surface with PC5-2 peptide, as shown in FIG. 21.

### Method of Preparing Nanoparticle Carrier

Different ratios of synthesized PLGA-mPEG and PLPGA-AuNCs were combined to form nanoparticles for observing the targeting ability of PEG against anti-PEG antibody as well as tracking ability of AuNCs images. Composite ratios were illustrated as in Table 1 below.

TABLE 1
Nanoparticle Groups

| Polymer | Ratio |
|---|---|
| PLGA | control |
| PLGA-mPEG | control |
| PLGA-AuNCs:PLGA-mPEG | (2:1) |
| PLGA-AuNCs:PLGA-mPEG | (1:1) |
| PLGA-AuNCs:PLGA-mPEG | (1:2) |

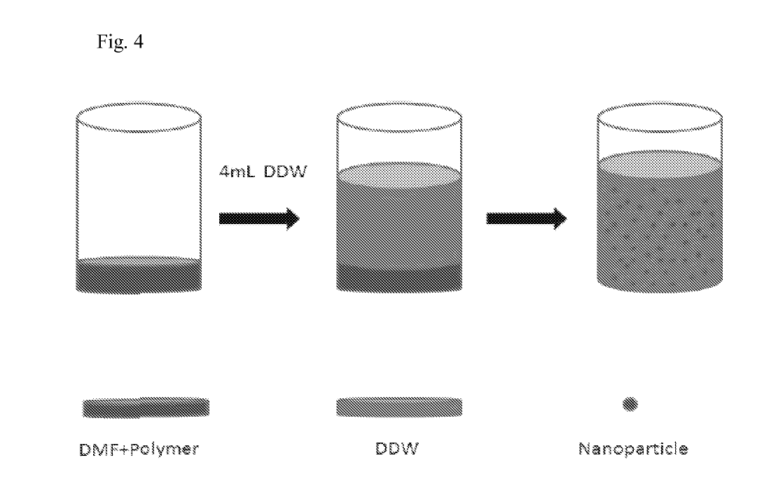

Nano-test was proceeded with the ratios shown in Table 1 and the concentration of the nanoparticle solution during the test was approximately 25 mg/5 ml. First, total of 25 mg of materials were weighed and obtained, dissolved in 1 ml of DMF solvent with evenly stir for combination. Nano-step was then proceeded with 1:4 ratio, 4 ml of DD water was obtained and added quickly at once into the reaction flask. Because the volume of DD water was larger than DMF solvent, phase transition effect was generated, resulting in physical cross-link of the hydrophobic ends of PLGA structure contained in the solution that formed nanoparticles, as shown in FIG. 4.

### NMR Nuclear Magnetic Resonance Test

0.5 mg of dry PLGA, mPEG, PLGA-mPEG and PLGA-AuNCs powders were obtained and dissolved in 0.6 ml of chloroform-d, placed inside the NMR tube, and ¹H NMR nuclear magnetic resonance spectrometer (Varian Gemini-200) was used to proceed ¹H NMR nuclear magnetic resonance spectrum analysis. Location of the signal of the functional groups of the polymer materials was used to validate the synthetic structure.

### Transmission Electron Microscopy (TEM) Observation

TEM was utilized to observe the pattern and particle size of AuNCs and to observe the distribution of AuNCs in PLGA-AuNCs nanoparticles. AuNCs and PLGA-AuNCs nanoparticles were placed in droplet onto the TEM copper grid, air-dried for 2 days, and lastly placed inside the TEM for observation.

### Fluorescent Capability Test

Fluorescent spectrometer was used to effectively test light emission effect of AuNCs and composite nanoparticles. Emission wavelength band and intensity could be obtained by this test, providing reference for assessing condition design of subsequent in vitro cellular endocytosis test and live molecular images.

### Analysis of Particle Size of AuNCs and Nanoparticles

Particle size of the nanoparticle in the solution after completion of nano-preparation was analyzed using ζ potential-particle size analyzer (Zetasizer 3000HSA). Test was repeated three times to validate particle size and investigate effect from different composite ratios.

### Assessment of Cytotoxicity and Cell Endocytosis Capability

Assessment of cytotoxicity and cell endocytosis ability of AuNCs and nanoparticles with different composite ratios was done by using HeLa (ATCC®CCL-2™) and 3T3 (ATCC®CRL-2593) cell lines obtained from ATCC (ATCC was a private, nonprofit biological resource center (BRC)) as the assessment objects. HeLa cell line is the cancer cell derived from human cervical cancer. The present study is to investigate the targeting ability of nanoparticles against anti-PEG antibody. Antibody gene was cloned into HeLa cells, and HeLa cells were tested in this cytotoxicity assay.

HeLa cell line and 3T3 cell line were thawed and cultured with the basic culture medium (low glucose DMEM (purchased from GIBCO), supplemented with 10% fetal bovine serum (FBS, purchased from Biological Industries), 1.5 μl/ml of NaHCO3 and 5% penicillin/streptomycin (P/S)) under 37° C. and 5% CO2 inside an incubator. Cells were subcultured after growth.

HeLa cells, 3T3 cells (5000 cells/200 μl), AuNCs and nanoparticles with different composite ratios (20 μg/ml) were cultured in the basic culture medium for 3 and 5 days, followed by cell growth viability test using MTS assay. First, the basic culture medium containing AuNCs or nanoparticles was removed. PBS was used to wash twice. 100 μl of culture medium was added. Finally, 20 μl of MTS agent was added to react for 1 hour. An ELISA reader was used for detection at 490 nm wavelength.

### Functional Test of In Vitro Targeting

The present study used the anti-PEG antibody developed in the laboratory of Professor Tian-Lu Cheng at the Department of Biomedical Science and Environment Biology in the College of Life Science at Kaohsiung Medical University to perform targeting ability test on the nanoparticles with PEG modification. First, the anti-PEG antibody was cloned into cancer cells, the nanoparticles were modified with PEG antibody, making them possess targeting ability with binding specificity against the antibody or receptor on the surface of cancer cells.

Colloidal solution of freshly synthesized composite nanoparticles was obtained and diluted in 1, 0.5, 0.25 and 0.125 folds to be the stand-by colloidal solution. In addition, pure PLGA nanoparticles were prepared as control group. 20 mg of PLGA raw material was obtained and prepared using the same nano-precipitation method to generate PEG-free PLGA nano-colloidal solution as control group in the experiment. Control group of PLGA nano-colloidal solution was diluted in 10, 30, 90 and 270 folds and added into the 96-well culture plate pre-coated with anti-PEG antibody. 50 μl/well of prepared nano-colloidal suspension material was placed into the culture plate and incubated in room temperature for 1 hour. PBS washing was proceeded, then secondary antibody was added and incubated in room temperature for 1 hour. PBS washing was continued, then streptavidin-HRP was used for labeling in room temperature for 1 hour, followed by PBS washing. Lastly ABTS was added in a light-proof environment to react in room temperature for 15 minutes, then detection of absorbance at OD 405 nm began.

### Cell Endocytosis Assay

20 mg of polymer material was dissolved in DMF and stirred. After complete dissolving, 2.5 mg of fluorescein isothiocyanate (FITC) (purchased from Sigma-Aldrich) was added and stirred continuously while light should be avoided. Finally DD water was added instantly, followed by continuous stir for 1 hour. The solution was taken and placed into the dialyzing membrane with molecular weight cut-off (MWCO) of 1,000. DD water was used for dialysis for 2 hours, followed by 4 hours of dialysis using DMF and lastly about 2 days of dialysis using water. Prepared nano-colloidal solution material was added onto HeLa cells with and without anti-PEG antibody for 30 minutes, PBS buffer and DD water were used to wash, then DAPI (4′,6-diamidino-2-phenylindole) (purchased from Sigma-Aldrich) was used for labeling, followed by fluorescent microscopy imaging.

### Live Molecular Image Tracking Validation

The present method was to investigate fluorescent image tracking ability of AuNCs and composite nanoparticles in live animals using IVIS system (IVIS 200 Imaging System) for testing. 0.5-1 ml of AuNCs and (1:1) composite nanoparticles were injected on the dorsal side of BALB/c strain mice after epilation for comparison.

### Micro CT Imaging

Micro CT for animal study use was used to examined AuNCs and PLGA-AuNCs nanoparticles. Differences compared with PBS were observed under parameters of 140 keV, 250 mA and 0.67 thickness per slice for 256 slices.

### Bicinchoninc Acid Procedure Assay (BCA Assay)

The principle is that the structure of peptide bonds in protein molecules can form a complex with Cu²⁺ in basic environment and reduce Cu²⁺ into Cu⁺. BCA reagent can specifically bind to Cu⁺ to form a stable colored composite, yielding maximum absorbance at 562 nm. The intensity of the color of the composite has positive correlation with the concentration of the protein, therefore, the amount of the protein can be detected according to the absorbance value.

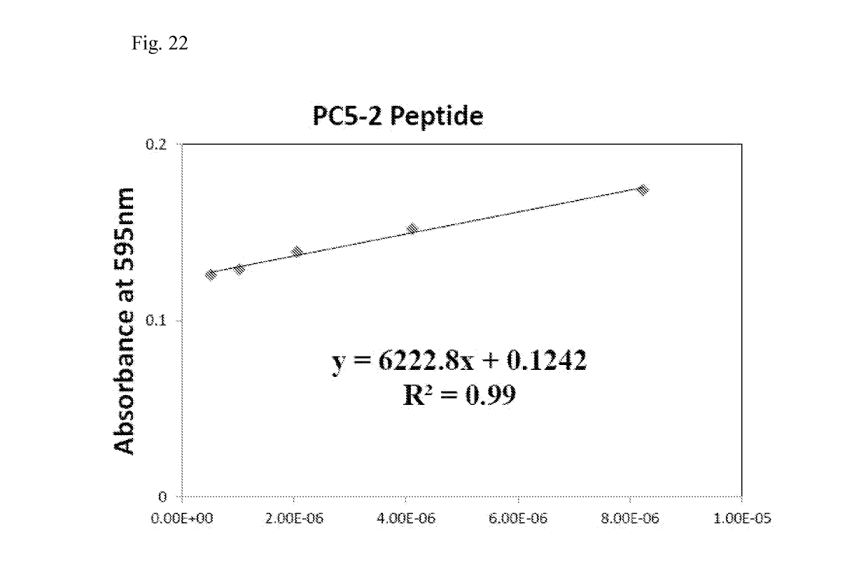

If the surface of the nanoparticles can be modified with PC5-2 Peptides was investigated and tested. Through BCA assay test result, it was confirmed that the surface of nanoparticles had been modified with carbonic anhydrase inhibitors, and through calibration curve calculation, the grafting rate was calculated as 87.2%, as shown in FIG. 22.

### Results:

### Validation of Synthesis of AuNCs

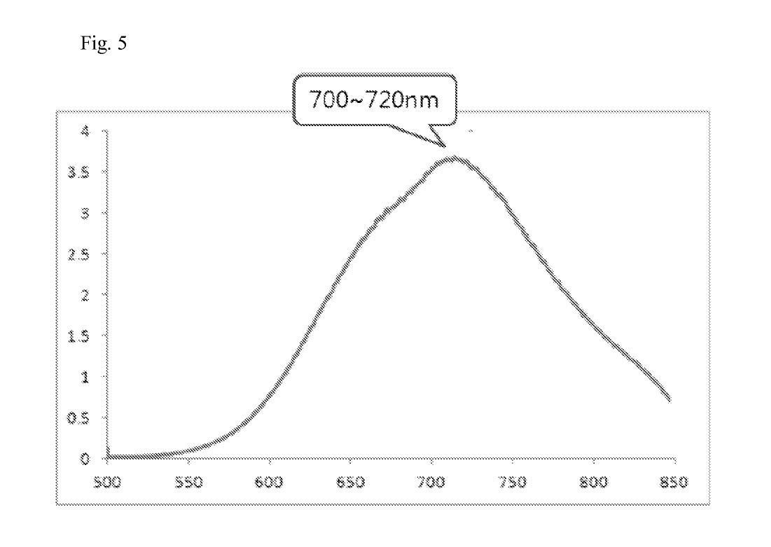

First, fluorescent spectrometer was used to test AuNCs synthesized with (±)-α-diaphorase. AuNCs were excited with excitation wavelength at 310 nm and the emission signal at 500 nm is collected to yield result shown in FIG. 5. It was known from FIG. 5 that emission from AuNCs was within a wide wave band wherein the peak emission region was located at 700-720 nm, falling behind 600 nm. This wave band region not only reduced the background noise at emission wave band coming from biomatrix, but also was effectively observable within the tracking range of cell fluorescence, meeting the experimental needs on the wave band available for cellular observation or image tracking in vivo. If AuNCs were placed under visible light and a UV light bulb, significant difference could be observed by eyes. AuNCs were present as dark brown colloidal solution under a fluorescent lamp while generation of red light could be observed by eyes when transferring AuNCs into UV light for observation. Through a simple fluorescent test, it was validated that the present invention synthesized the AuNCs available for fluorescent image tracking.

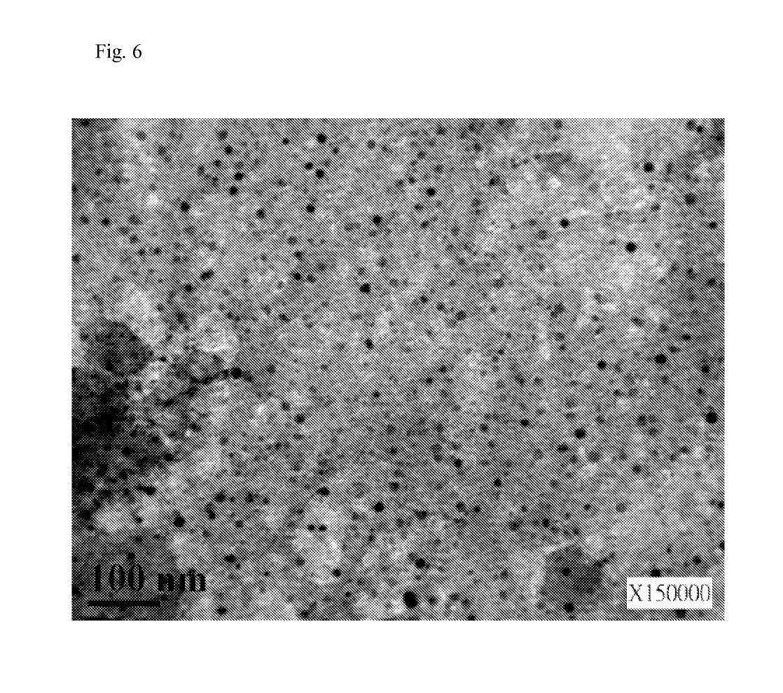

After further diluting the AuNCs, TEM transmission electron microscope was used to observe the shape and particle size of the AuNCs. It is shown in FIG. 6 that the AuNCs were present as round particles with the particle diameter approximately below 10 nm.

### Assessment of Particle Size of the Composite Nanoparticles

It was known from Table 2 below that the size of the nanoparticles averaged at 118 nm with the largest as 132 nm and smallest as 91.3 nm. It was known from the table that the higher proportion the PLGA-mPEG was, the smaller the nanoparticles formed were. In contrast, the higher proportion the PLGA-AuNCs was, the larger the nanoparticles formed were. Further investigating showed that the hydrophilic property of PEG trended to reduce the particle diameter of PLGA-based nanoparticles.

TABLE 2
Distribution of particle diameter of the composite nanoparticles

| | SIZE (nm) | PDI |
|---|---|---|
| PLGA | 112.6 | 0.2 |
| PLGA-mPEG | 127.3 | 0.2 |
| PLGA-AuNCs:PLGA-mPEG(2:1) | 132.3 | 0.3 |
| PLGA-AuNCs:PLGA-mPEG(1:1) | 127.1 | 0.2 |
| PLGA-AuNCs:PLGA-mPEG(1:2) | 91.3 | 0.2 |

### Fluorescent Test on Composite Nanoparticles

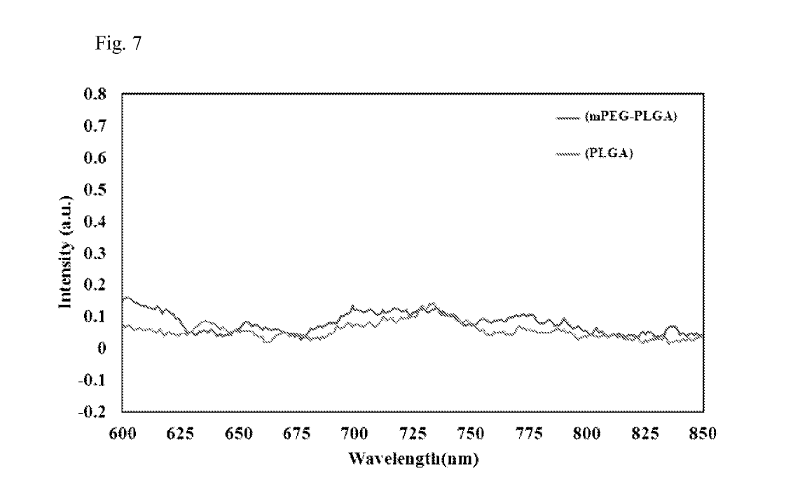

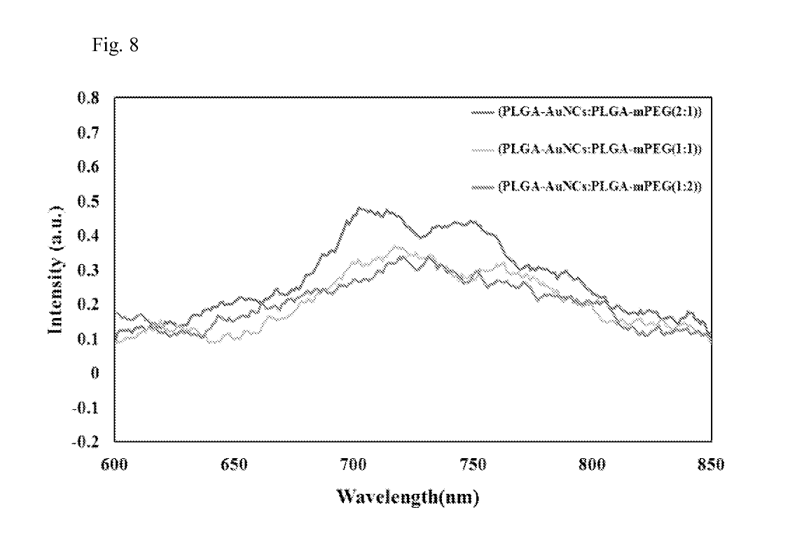

Nanoparticle colloidal solution was placed under fluorescent lamp and UV light to observe, it was visible by eyes that nanoparticle colloidal solution of PLGA and PLGA-mPEG was present as pure white with light transparency under fluorescent lamp but did not generate fluorescence under UV light. Fluorescent spectrometer was further used for testing and investigating and was shown in FIG. 7. It was evidenced in FIG. 7 that PLGA and PLGA-mPEG do not generate emission signal of fluorescent light within wave band of 700-720 nm, thus confirming that PLGA and mPEG did not affect the fluorescent result of the AuNCs. Similarly, when 3 groups, PLGA-AuNCs:PLGA-mPEG (2:1), PLGA-AuNCs:PLGA-mPEG (1:1) and PLGA-AuNCs:PLGA-mPEG (1:2), were placed under fluorescent lamp for observation, compared with PLGA and PLGA-mPEG, it was noticeable that the color of the colloidal solution was more toward brown and that the higher proportion PLGA-AuNCs were, the more brownish it was. When transferred under UV light, the fluorescent emission level could be observed as PLGA-AuNCs:PLGA-mPEG(2:1)>PLGA-AuNCs:PLGA-mPEG(1:1)>PLGA-AuNCs:PLGA-mPEG(1:2). FIG. 8 was yielded after analysis by fluorescent spectrometer. A significant peak at emission wave band of 700-720 nm was shown in FIG. 8, coincident with the location of the peak shown in FIG. 5, thus further confirming successful synthesis of PLGA-AuNCs nanoparticle.

### Cell Viability Test of AuNCs

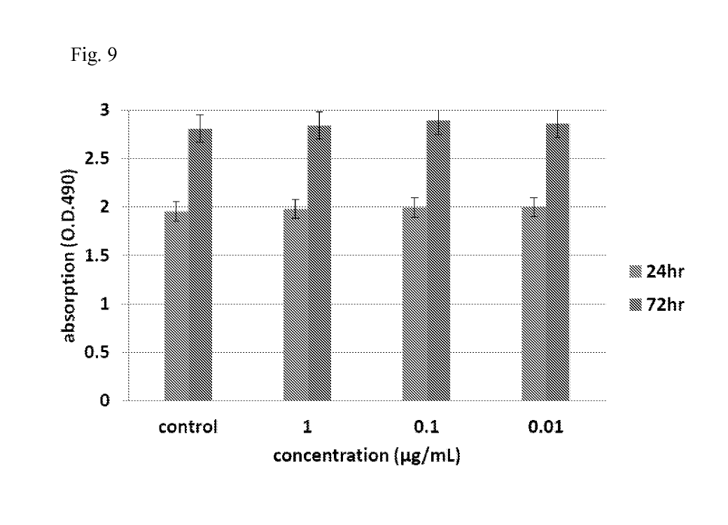

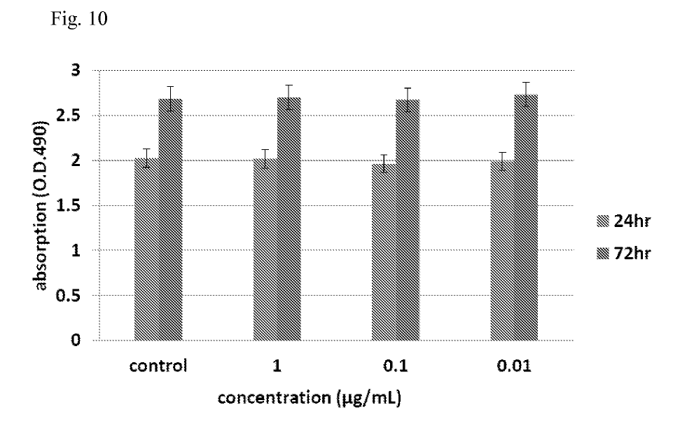

AuNCs were diluted in 1, 10 and 100 folds and incubated with HeLa cell line and 3T3 cell line respectively in a 96-well culture plate (5,000 cells/200 μl), the control group was cells only. 24 hours and 72 hours of observation were proceeded followed by MTS assay, the absorbance detected represented mitochondrial activity as well as the number of viable cells indirectly. It was shown in FIG. 9 and FIG. 10 that the cells continued to grow in parallel to the control group in 72 hours versus 24 hours after addition of AuNCs. This result confirmed that AuNCs did not generate inhibiting or poisoning effect to normal or cancer cells.

### Cellular Activity Test of Composite Nanoparticles

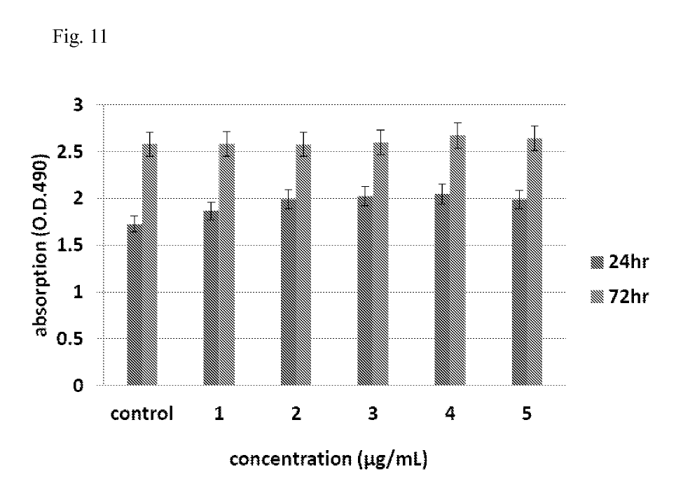

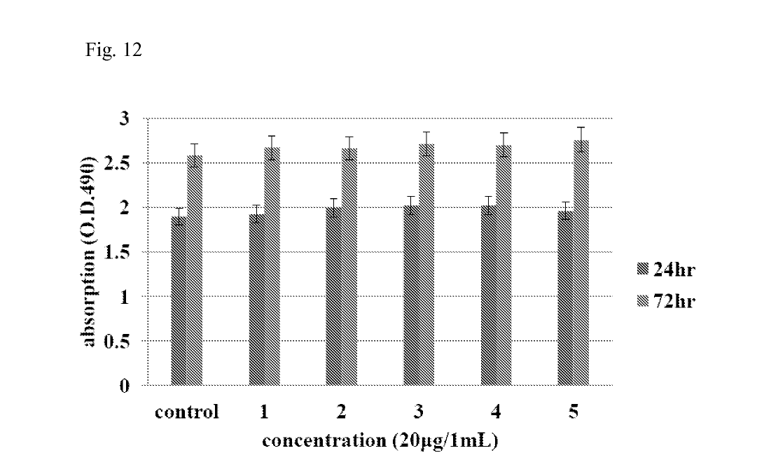

Nanoparticles were diluted to approximately (20 μg/ml) of concentration and incubated with HeLa cell line and 3T3 cell line respectively in a 96-well culture plate (5,000 cells/200 μl), the control group was cells only. 24 hours and 72 hours of observation were proceeded followed by MTS assay. It was shown in FIG. 11 and FIG. 12 that the cells continued to grow in parallel to the control group in 72 hours versus 24 hours after addition of different nanoparticles. This result confirmed that nanoparticles did not generate inhibiting or poisoning effect to normal or cancer cells.

### ELISA Assay for Binding Specificity of Nanoparticles Against Anti-PEG Antibody

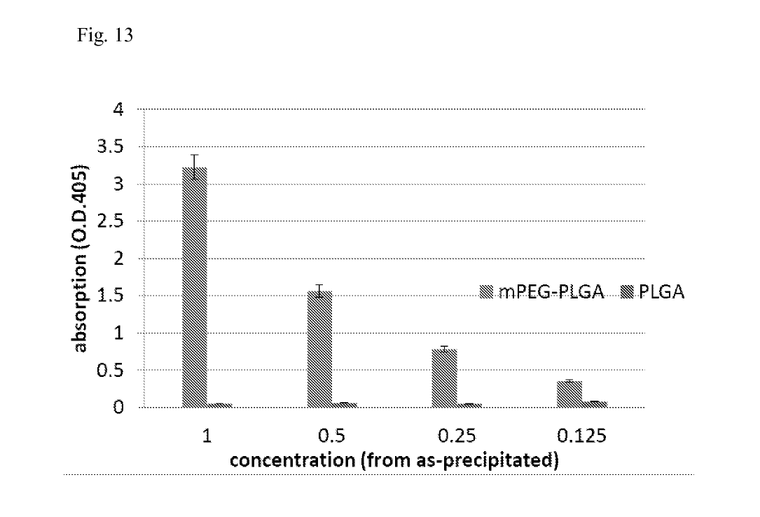

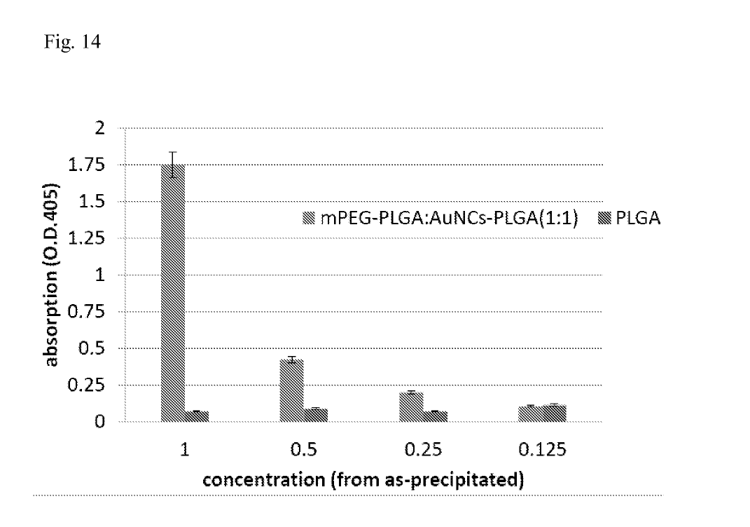

ELISA was used to test binding specificity of PLGA-mPEG and PLGA-AuNCs:PLGA-mPEG (1:1) nanoparticles as well as control PLGA nanoparticles against anti-PEG antibody. In the experimental results in FIG. 13 and FIG. 14, PLGA-mPEG nanoparticles and anti-PEG antibody had high binding specificity to each other. In contrast, PLGA nanoparticles used as control group showed no difference compared with blank control group, proving that the PLGA-mPEG nanoparticles in the present invention possessed binding specificity against anti-PEG antibody. By cross-relating FIG. 13 and FIG. 14, it was known that PLGA-AuNCs:PLGA-mPEG (1:1) composite nanoparticles could still possess binding specificity against anti-PEG antibody despite its half concentration of PLGA-mPEG, further confirming the targeting specificity possessed by the composite nanoparticles.

### In Vitro Fluorescent Microscopy Examination of Cellular Endocytosis

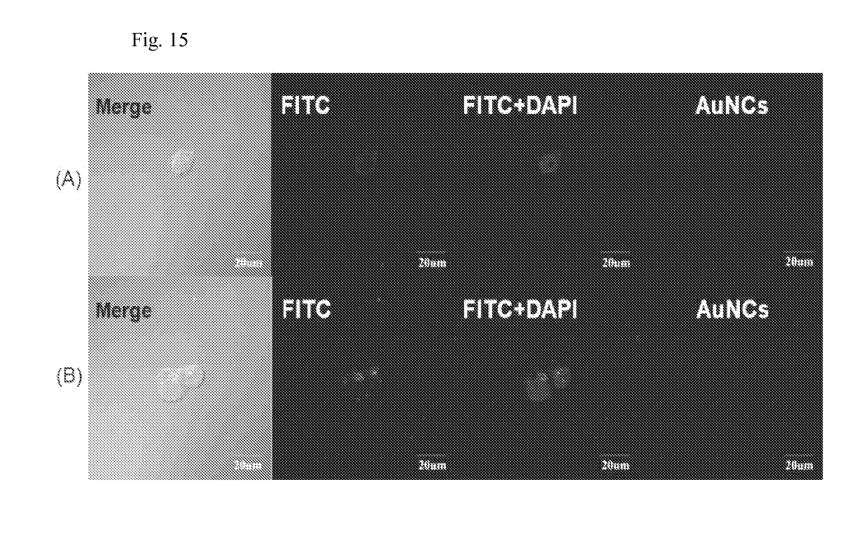

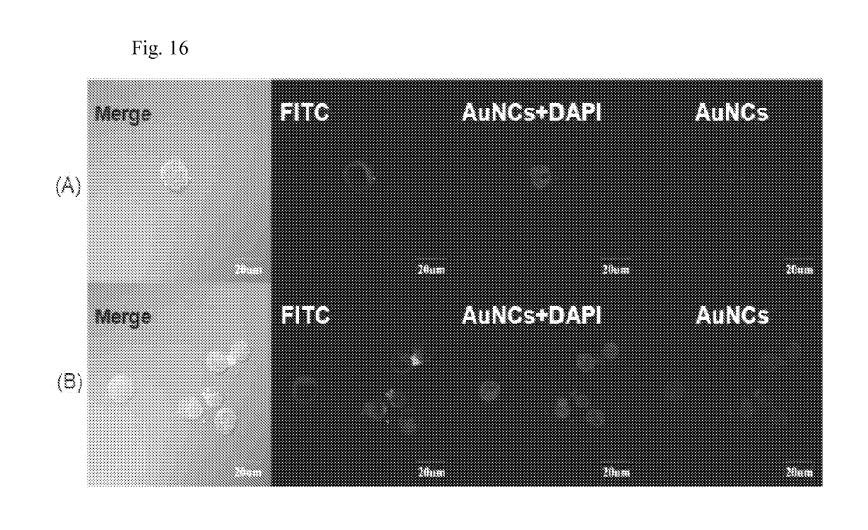

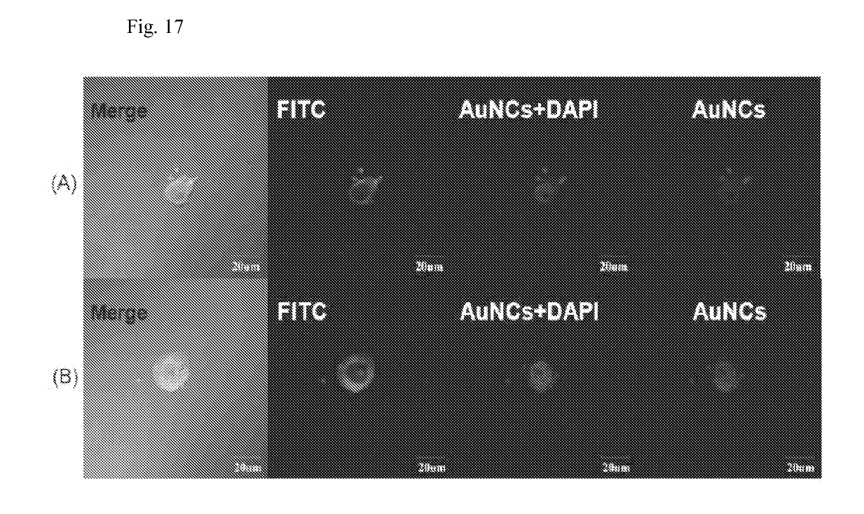

Three types of nanoparticles, PLGA, PLGA-AuNCs:PLGA-mPEG (1:2) and PLGA-AuNCs:PLGA-mPEG (1:1), were used on HeLa cells (A) without and (B) with anti-PEG antibody to conduct functional tests on encapsulation, targeting and image tracking of the nanoparticles. FITC was used as a drug model to be encapsulated into nanoparticles. After being nano-prepared and purified, it was added to the cellular endocytosis assay with 30 minutes of incubation time given, followed by PBS washing and fixation. Cell nuclei were labeled with DAPI in order to differentiate location of fluorescence. Lastly, fluorescent images were observed through a confocal laser microscope. FITC-green fluorescence, DAPI-blue fluorescence and AuNCs-red fluorescence were present respectively in the images. In the experimental result, FIG. 15 showed PLGA nanoparticles encapsulating FITC. By the relative location with DAPI, it was confirmed that nanoparticles can effectively enter into the cytoplasm after 30 minutes. Compared with FIG. 16 and FIG. 17, accumulation of FITC was lower and red fluorescence was absent in the images. This was caused mostly by the fact that nanoparticles formed by pure PLGA did not have binding specificity against anti-PEG antibody, thus its accumulation was not obvious compared to the PEG-modified nanoparticles. Red fluorescent images were non-existent because of absence of AuNCs modification. Comparing FIG. 16 with FIG. 17, red fluorescent images yielded by AuNCs at 700-720 nm were obvious, the red fluorescent images of PLGA-AuNCs:PLGA-mPEG (1:1) was larger with significant amount of accumulation compared with PLGA-AuNCs:PLGA-mPEG (1:2). From the light intensity of accumulated FITC, it was shown in FIG. 17 that significantly differential accumulation was seen between HeLa cells without anti-PEG antibody and HeLa cells with anti-PEG antibody in the same testing period, owing to the difference in PEG against anti-PEG antibody. The differential accumulation could be evidenced even more obvious at the red fluorescent wave band of AuNCs. The experimental results proved that PLGA-AuNCs:PLGA-mPEG composite nanoparticles through specific targeting ability of PEG structure against anti-PEG antibody developed faster and higher accumulation of nanoparticles and possessed function of encapsulating drugs to carry them into the cytoplasm and targeting via anti-PEG antibody, and AuNCs on the nanoparticles could be used as an imaging probe for image tracking.

In Vitro Cellular Endocytosis Assessment of the PC5-2 Peptide through Fluorescent Microscope

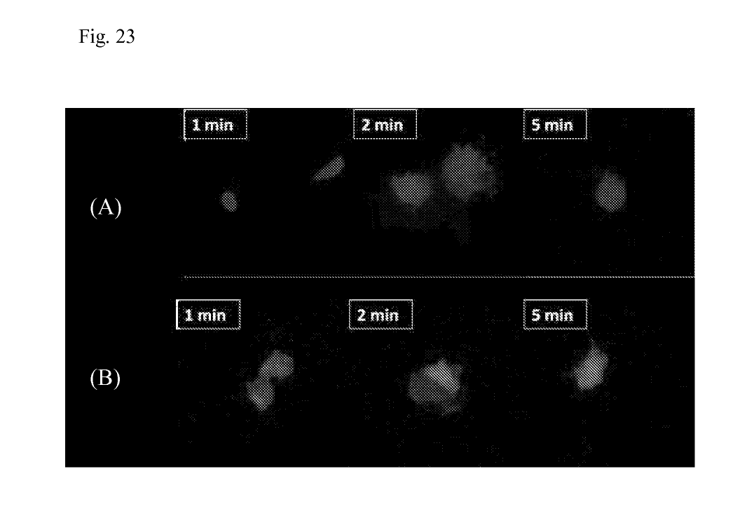

PLGA-AuNCs:PLGA-PEG:PC5-2 Peptide nanoparticles were used to test the ability of nanoparticle encapsulation, targeting and image tracking on A549 and 3T3 cells. Materials were processed with nanotechnology and purified, added to proceed cellular endocytosis assay, incubated for 5 minutes given, washed with PBS and proceeded with fixation. DAPI was used to label nucleus for differentiating fluorescent locations. Finally, the fluorescent images were observed through Laser Scanning Confocal Microscopy (LSCM). As shown in FIG. 23, DAPI and AuNCs were illustrated as blue light and red light respectively in the image. By observing the related locations of PLGA-AuNCs:PLGA-PEG:PC5-2 peptide nanoparticles and DAPI, it was confirmed in the experimental results that nanoparticles could effectively enter the cytoplasm after 5 minutes.

### In Vivo Molecular Fluorescent Image Tracking

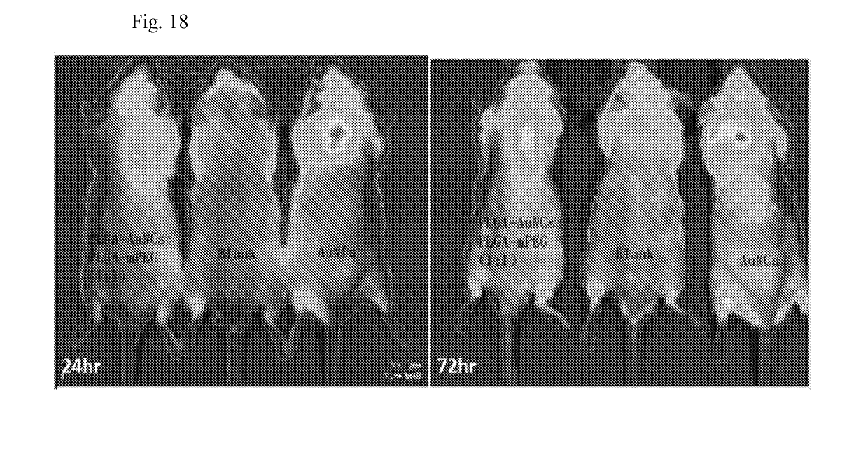

IVIS system was further utilized to conduct in vivo fluorescent image tracking test to validate if PLGA-AuNCs:PLGA-mPEG composite nanoparticles possessed fluorescent imaging capability in the in vivo model. PLGA-AuNCs:PLGA-mPEG (1:1) composite nanoparticles and AuNCs were aspirated into the syringes separately and placed under IVIS, excited with 465 nm and their emission with wave band after 600 nm were collected. It was found that AuNCs owing to lack of PLGA encapsulation produced stronger fluorescent intensity than PLGA-AuNCs:PLGA-mPEG (1:1) composite nanoparticles. 0.5-1.0 ml of AuNCs and PLGA-ANCs:PLGA-mPEG (1:1) composite nanoparticles were injected on the dorsal side of BALB/c strain mice after epilation for comparison. Observation at 24 hours and 72 hours after injection was proceeded. It was shown in FIG. 18 that the fluorescent intensity of AuNCs was stronger than PLGA-AuNCs:PLGA-mPEG (1:1) composite nanoparticles. This result was identical to the pre-injection result, nevertheless it also proved that AuNCs grafted on PLGA through modification still possessed fluorescent development potential. It was also proved from FIG. 18 that AuNCS and PLGA-AuNCs:PLGA-mPEG (1:1) composite nanoparticles did not develop any discomfort, neither did they cause death after injection into animal model, and that the fluorescence stayed more than 3 days, thus they were suitable to be the material for image tracking and pharmaceutical therapy use.

### In Vivo Image Assessment of PC5-2 Peptide Target Tracking

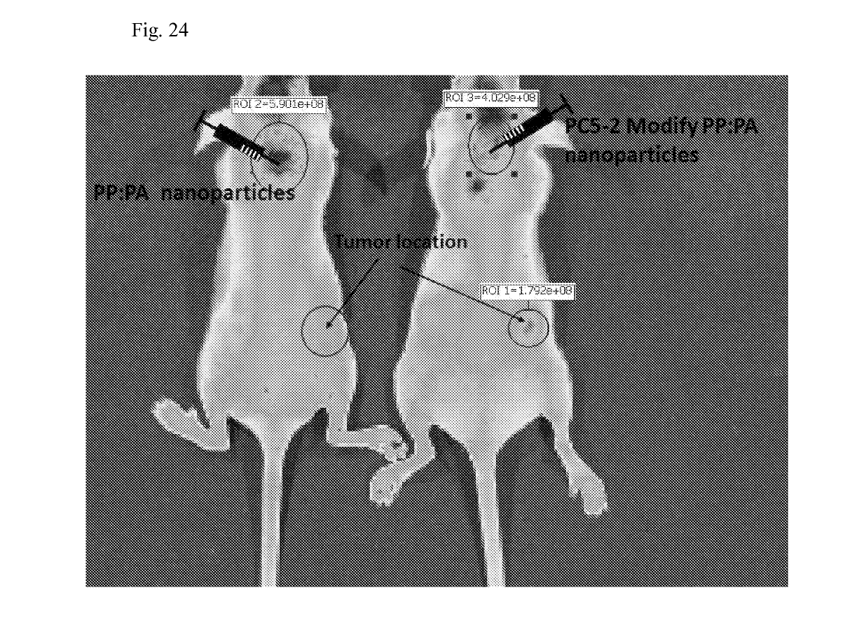

Utilizing PC5-2 peptide-modified composite nanoparticles and unmodified composite nanoparticles as control group, respective groups were injected into A549 cell line-induced tumorigenic immunocompromised mice (nude mice BALB/c nu/nu). Through in vivo fluorescent image observation, more accumulated amount of PC5-2 peptide-modified nanoparticles at the tumor site was found after 6 hours compared with the control group, as shown in FIG. 24. This result provided evidence that modification of the composite nanoparticles with targeting peptide(s) or protein(s) could enhance the accuracy and time efficiency of drug delivery, effectively reach treatment goals and be able to track the drug delivery in vivo with real-time image information, meeting the designs on drug delivery, targeting and image tracking in the present experiments.

### Micro CT Examination

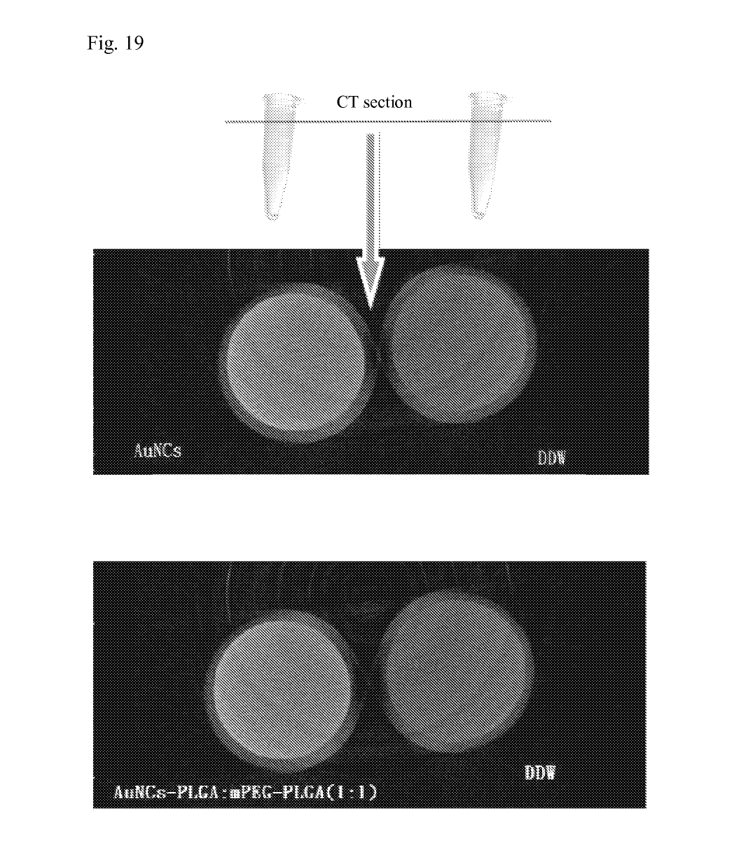

The present study was to test the possibility of applying AuNCs on human body in the medicine in the future to replace the currently available contrast agents. AuNCs and DD water (DDW) were obtained with 0.6 ml micro test tube and placed into micro CT for animal study use. Gray scale images generated from AuNCs and DDW were shown in the top picture of FIG. 19 where significantly AuNCs possessed higher value in gray scale. It was shown from the bottom picture of FIG. 19 that the gray scale image developed by AuNCs similarly had higher value in gray scale. Moreover, compared with the top picture, no significant difference was shown between AuNCs and PLGA-AuNCs:PLGA-mPEG (1:1) composite nanoparticles, unlike the difference in fluorescence in the fluorescent images produced by PLGA-encapsulated AuNCs. This proved that AuNCs or PLGA-AuNCs:PLGA-mPEG (1:1) composite nanoparticles were helpful to CT imaging.

Taken together, the three functions of PLGA-AuNCs:PLGA-mPEG composite nanoparticles on drug encapsulation, specific targeting and image tracking would be helpful to future cancer chemotherapy on encapsulating hydrophobic drugs for chemotherapy, accumulating the carried nanoparticles at the affected area through specific antibody targeting function and with the AuNCs being used as imaging probe for image tracking during treatment.

One skilled in the art readily appreciates that the present invention is well adapted to carry out the objects and obtain the ends and advantages mentioned, as well as those inherent therein. The nanoparticle carriers, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A nanoparticle carrier comprising hydrophobic molecules bonded with gold nanoclusters (AuNCs) and hydrophobic molecules bonded with hydrophilic molecules, wherein the hydrophilic molecules are located in the outer layer of the nanoparticle and the AuNCs are encapsulated inside the nanoparticle.

2. The nanoparticle carrier of claim 1, wherein the hydrophobic molecules are selected from poly(lactide-co-glycolide) (PLGA), polycaprolactone (PCL), polyvalerolactone (PVL), polylactic acid (PLA), polybutyrolactone (PBL), polyglycolide (PLG), or polypropiolactone (PPL).

3. The nanoparticle carrier of claim 1, wherein the hydrophilic molecules are selected from polyethylene glycol (PEG), hyaluronic acid, poly(glutamic acid) (PGA), dextran, chitosan, or gelatin.

4. The nanoparticle carrier of claim 1, wherein the hydrophobic molecules bonded with AuNCs are PLGA-AuNCs and the hydrophobic molecules bonded with hydrophilic molecules are PLGA-mPEG.

5. The nanoparticle carrier of claim 1, wherein the particle size of the nanoparticle ranges from 20 to 300 nm.

6. The nanoparticle carrier of claim 1, which further encapsulate an active material inside.

7. The nanoparticle carrier of claim 6, which the active material is selected from drugs, proteins, polysaccharides, radioactive substances, growth factors, or genes.

8. The nanoparticle carrier of claim 1, wherein the hydrophilic molecules are further bonded with a functional molecule.

9. The nanoparticle carrier of claim 8, wherein the functional molecule is a targeting molecule with targeting capability.

10. A method of manufacturing the nanoparticle carrier of claim 1, comprising (a) dissolving hydrophobic molecules bonded with gold nanoclusters (AuNCs) and hydrophobic molecules bonded with hydrophilic molecules in an organic solvent to yield a mixture; and (b) adding water into the mixture.

11. The method of claim 10, wherein the hydrophobic molecules are selected from poly(lactide-co-glyco lide) (PLGA), polycapro lactone (PCL), polyvalero lactone (PVL), polylactic acid (PLA), polybutyrolactone (PBL), polyglycolide (PLG), or polypropiolactone (PPL).

12. The method of claim 10, wherein the hydrophilic molecules are selected from polyethylene glycol (PEG), hyaluronic acid, poly(glutamic acid) (PGA), dextran, chitosan, or gelatin.

13. The method of claim 10, wherein the hydrophobic molecules bonded with AuNCs are PLGA-AuNCs and the hydrophobic molecules bonded with hydrophilic molecules are PLGA-mPEG.

14. The method of claim 13, wherein the mixing ratio of PLGA-AuNCs and PLGA-mPEG ranges from 1:10 to 10:1.

15. The method of claim 10, wherein the organic solvent is dimethylformamide (DMF), dimethyl sulfoxide (DMSO), tetrahydrofuran (THF), acetone, dichloromethane, or chloroform.

16. The method of claim 10, wherein the ratio of the water and the organic solvent in volume is 1:20 to 20:1.

17. The method of claim 10, wherein the step (a) further comprises addition of an active material into the mixture.

18. The method of claim 17, wherein the active material is selected from drugs, proteins, polysaccharides, radioactive substances, growth factors, or genes.

19. The method of claim 10, wherein the hydrophilic molecules in step (a) are further bonded with a functional molecule.

20. The method of claim 19, wherein the functional molecule is a targeting molecule with targeting capability.
